(19) United States
(12) Patent Application Publication
(10) Pub. No.: US 2008/0245684 A1
(43) Pub. Date: Oct. 9, 2008

(54) Toiletry & Sundries Attachment And Containment Device

(76) Inventor: Dennis Yeatman, San Diego, CA (US)

Correspondence Address: Wayne M. Chancellor, 455 Muirfield Circle, Louisville, CO, 80027, US

(21) Appl. No.: 12/098,774

(22) Filed: Apr. 7, 2008

Related U.S. Application Data
(60) Provisional application No. 60/921,938, filed on Apr. 5, 2007.

(51) Int. Cl. B65D 69/00; A45C 15/00; B65D 85/00; A61B 19/00

(52) U.S. Cl. 206/233; 206/38; 206/397; 206/389; 206/438

(57) ABSTRACT

Provided is an toiletry and sundries attachment and containment device for interfacing a container or other containment means with a tissue dispensing apparatus found in public and private restrooms. The device includes one or more tabs for attaching to the container, and for attaching the container to the tissue dispensing apparatus. The tabs may be rigid, semi-rigid or flexible, and may be manufactured from a variety of materials including paper, plastic and metal. The attachment mechanisms or schemes may be any of a number of attachment mechanisms to include: mechanical fasteners, glue, Velcro®, adhesives strips, tape, etc. The container may be permanently or removably attached to the tabs, and hence the tissue dispensing apparatus, and may contain tissues, wet wipes, soaps, as well as other toiletries and sundries for use in a restroom.

### FIELD OF THE INVENTION

This invention relates generally to toiletry item containers. More particularly, to a device for removably or permanently attaching a container having toiletries and/or various other sundries to a tissue dispensing device such as a toilet paper or paper towel roll/dispenser. This invention claims priority to provisional application Ser. No. 60/921,938, “Toiletry & Sundries Attachment and Containment Device”, filed Apr. 5, 2007.

### BACKGROUND

Various toiletry item dispensers, to include those for containing and dispensing tissues, wet wipes, etc., exist currently. These dispensers/containers typically are stand alone items directed solely toward a single product. Many are one-time use products intended to be disposed of once the toiletry items are fully consumed. Further, these dispensers/containers are not, for the most part, designed to be integrated with other fixtures, containers, or devices found in public and private restrooms.

Persons of all age may have a need or desire to have both dry and wet wipe materials present in the restroom, along with other toiletry items. A simple, integrated device is needed which: attaches to existing fixtures in the restroom; is simple and easy to install; may be either disposable or reusable; and is cost effective to manufacture and distribute.

### SUMMARY

The methods and systems herein disclosed advance the art and overcome problems articulated above by providing a toiletry and sundries attachment and containment device that holds for dispensing tissues, wet wipes, etc., and integrates easily with a variety of tissue dispensing apparatus found in public and private restrooms.

In particular, and by way of example only, according to an embodiment, provided is a toiletry and sundries attachment and containment device including: a containing means for containing toiletry and sundries; and an interfacing means for interfacing the containing means with a tissue dispensing apparatus; wherein the containing means is suspended via the interfacing means in proximity to the tissue dispensing apparatus.

In yet another embodiment, provided is a toiletry and sundries attachment device including: a first tab having a first attachment mechanism for attaching to a tissue dispensing apparatus and a second attachment mechanism for attaching to a container; and a second tab having a first attachment mechanism for attaching to the tissue dispensing apparatus and a second attachment mechanism for attaching to the container.

In still another embodiment, provided is a toiletry and sundries attachment and containment device including: a container for containing toiletries and sundries; a first tab having a first attachment mechanism for interfacing with and attaching to a tissue dispensing apparatus, and a second attachment mechanism for interfacing with and attaching to the container; and a second tab having a first attachment mechanism for interfacing with and attaching to the tissue dispensing apparatus, and a second attachment mechanism for interfacing with and attaching to the container.

### BRIEF DESCRIPTION OF THE DRAWINGS

FIG. 1 is a plan view of a tab for attaching a toiletry and sundry items, and an integrated containment device, to a tissue dispensing apparatus, according to an embodiment;

FIG. 2 is a plan view of a tab for attaching a toiletry and sundry items, and an integrated containment device, to a tissue dispensing apparatus, according to an embodiment;

FIG. 3 is a plan view of a tab for attaching a toiletry and sundry items, and an integrated containment device, to a tissue dispensing apparatus according to an embodiment;

FIG. 4 is a perspective view of a toiletry and sundries attachment and containment device, according to an embodiment;

FIG. 5 is a perspective view of a toiletry and sundries attachment and containment device, according to an embodiment;

FIG. 6 is a side view of a tab for attaching a toiletry and sundry items, and an integrated containment device, to a tissue dispensing apparatus, according to an embodiment;

FIG. 7 is a plan view of a tab for attaching a toiletry and sundry items, and an integrated containment device, to a tissue dispensing apparatus, according to an embodiment;

FIG. 8 is a plan view of a toiletry and sundries attachment and containment device, according to an embodiment; and

FIG. 9 is a side view of a toiletry and sundries attachment and containment device, according to an embodiment.

### DETAILED DESCRIPTION

Before proceeding with the detailed description, it should be noted that the present teaching is by way of example, not by limitation. The concepts herein are not limited to use or application with one specific type of toiletry and sundries attachment and containment device. Thus, although the instrumentalities described herein are for the convenience of explanation, shown and described with respect to exemplary embodiments, the principles herein may be equally applied in other types of toiletry and sundries attachment and containment devices.

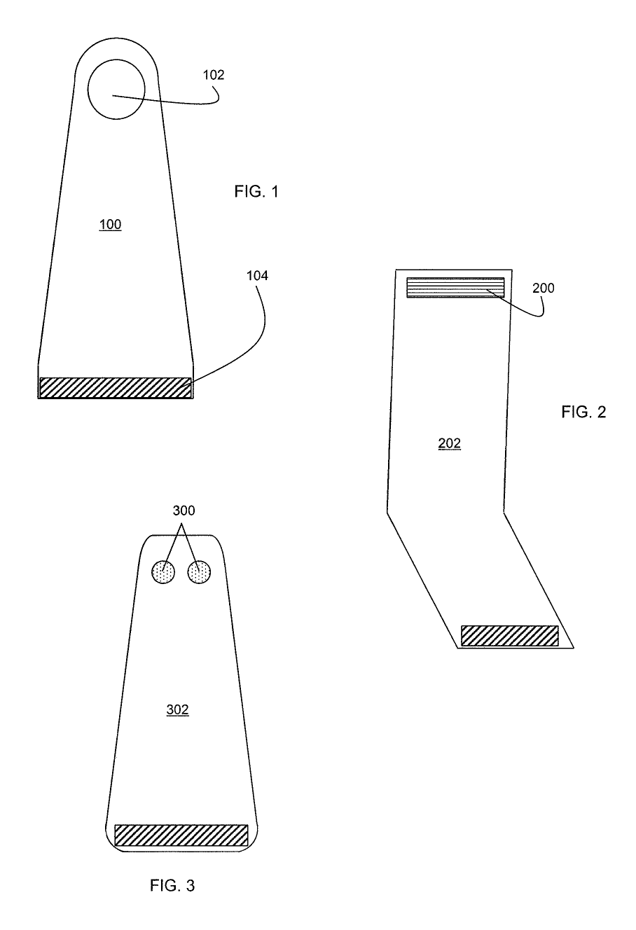

Referring initially to FIGS. 1-3, various tabs for interfacing a toiletry and sundries containment device, container, or containing means with a tissue dispensing apparatus or device are presented. For the purposes of this disclosure, toiletry and sundries may include, but are not limited to, items typically found in a public or private restroom or bathroom, such as tissue paper, paper towels, cloth towels, wet wipes, soaps, lotions, etc. A tissue dispensing apparatus may be any device or apparatus used primarily or secondarily for the dispensing of tissues, paper towels, cloth towels, etc. As noted in the FIGS. 1-3, the tabs may be of varying shape and size, and may include tapered or non-tapered ends, rounded and non-rounded or straight edges, as well as various methods for attaching the tab (and hence the toiletries, etc.) to the tissue dispensing apparatus. The tabs may be manufactured from any flexible, semi-flexible, semi-rigid or rigid material, such as paper, cardboard, plastic or metal, and may be reusable or disposable.

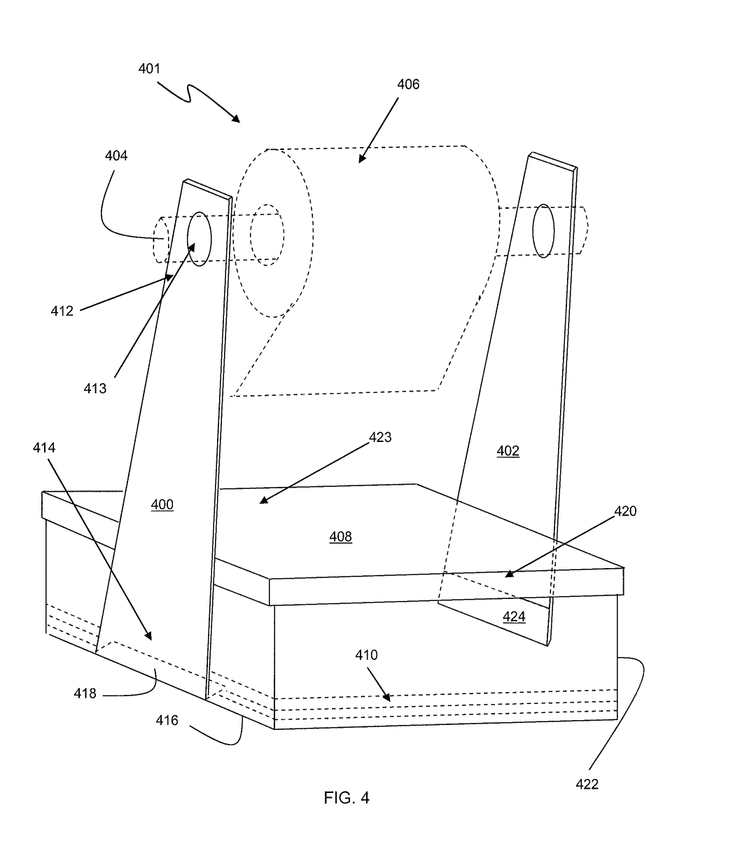

As shown in FIG. 1, a tab 100 may include an opening or slot 102 through which may be inserted one end of a tissue or paper towel roller. In this manner, the tab 100 (or tabs) and corresponding containment device are suspended substantially below, but in close proximity to, the roller. In the execution of the present invention, the roller is inserted through a first slot 102 in tab 100, and subsequently inserted through a second slot (see e.g. FIG. 4) of a second similar tab (see e.g. FIG. 4) such that the ends of the roller extend through and beyond the tabs.

Each tab, e.g. tab 100, may also include a means for attaching the tab to a container, containing means, or other containment device. Still referring to FIG. 1, the attachment mechanism 104 for attaching tab 100 to a container, containing means, or other containment device (not shown in FIGS. 1-3) may include, but is not limited to, an adhesive strip, a semi-permanent adhesive strip, a Velcro® strip, a mechanical fastener, glue, or any other means well known in the art for removably or permanently mechanically fastening one surface to another.

Referring now to FIGS. 2 and 3, the mechanisms or means 200 and 300 for interfacing the tabs 202 and 302 respectfully with a tissue dispensing apparatus may be an adhesive strip 200, mechanical fastening device such as Velcro® tabs 300, or any other mechanical means for removably, semi-permanently or permanently adhering or attaching the tabs, e.g. 202 and 302, and hence the container, to the tissue dispensing apparatus. These means for attachment may include, but are not limited to, an adhesive strip, a semi-permanent adhesive strip, a Velcro® strip, a mechanical fastener, and glue. In this way, the invention of the present disclosure may be interfaced with any type of tissue dispensing apparatus well known in the art, whether such device includes a roller or is simply a box or bin for holding and dispensing tissues. In at least one embodiment of the present disclosure, the tabs, e.g. tabs 100, 202 and 302, are permanently attached to a tissue dispensing apparatus, and a container, containing means, or containment device may be removed from the tabs and replaced. In yet another embodiment, the tabs are removably interfaced with the tissue dispensing apparatus, and the container is permanently attached to the tabs. Stated differently, the tabs are integral to the container, while being removably interfaced with the tissue dispensing apparatus. In yet another embodiment, both the attachment of the tabs to the tissue dispensing apparatus, and the attachment of the tabs to the container, is via a non-permanent attachment scheme. Stated differently, both the tabs and container are removable.

Referring now to FIG. 4, a toiletry and sundries attachment and containment device 401, in accordance with the present disclosure, is presented. As shown, two tabs 400 and 402 are positioned and aligned to interface with a roller 404 of a tissue dispensing or retaining apparatus or roll 406. In this manner, a container 408 containing, for example tissues or some form of wet wipe 410, is removably attached to the roller 404, and hence the tissue dispensing or retaining apparatus 406. Importantly, the container 408 may be disposable, replaceable, or reusable. Referring for a moment to tab 400, one end 412 interfaces with and attaches to the roller 404, via a hole or slot 413, while a second end 414 interfaces with and attaches to container 408. Specifically, in at least one embodiment, the second end 414 of tab 400 is attached (either removably, semi-permanently or permanently) to a bottom surface 416 of container 408 via an adhesive strip 418 or other mechanical attachment means. Alternatively, as shown with tab 402, one end 420 of tab 402 may be attached to a side 422 or top 423 of container 408 via an adhesive strip 424 or other mechanical attachment means. As can be appreciated by those skilled in the art, device 401 may be permanently or removably interfaced with tissue dispensing apparatus 406, and internal to device 401 container 408 may be permanently or removably interfaced with tabs 400 and 402. Apparatus 406, with roller 404, is just one of any number of a type of tissue/toiletry dispensing apparatuses well known in the art with which the invention of the present disclosure may interface.

In at least one embodiment of the present disclosure, tabs 400 and 402 may be flexible or semi-flexible, and may be folded or be bent such that the tabs 400, 402 fold over the top 423, or alternatively under the bottom 416, of container 408 during shipping and storage.

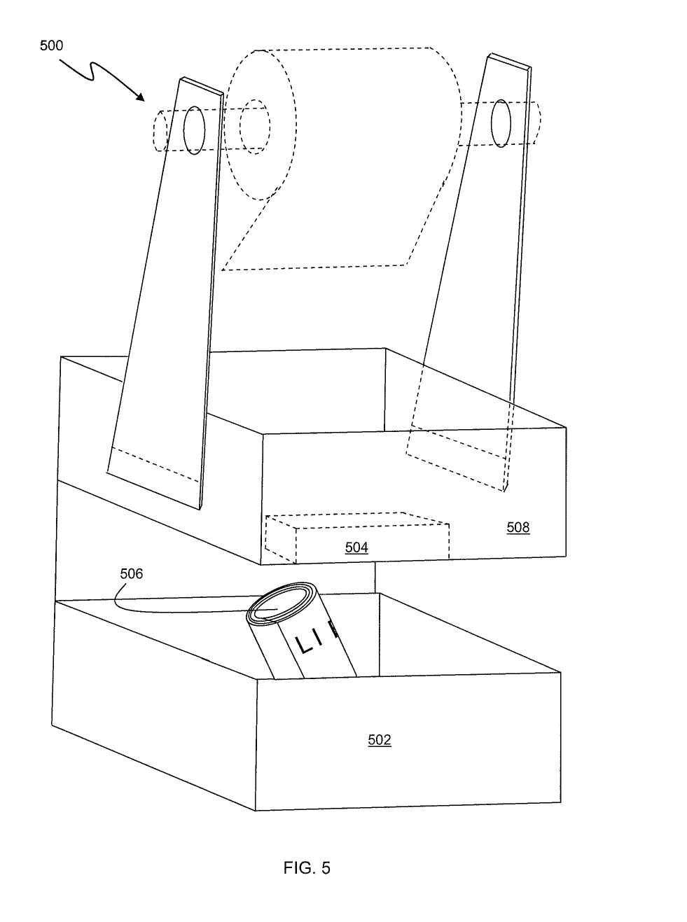

Turning now to FIG. 5, toiletry and sundries containment and attachment device 500 may include a tray 502 or other such device for containing and storing various items found in a bathroom, kitchen, etc, such as bars of soap 504 and a magazine 506. As contemplated by the present disclosure, any type of container, storage bin, tub, box, tray, or holder may be included in the present invention without departing from the intent thereof. As shown in FIG. 5, multiple containment devices (e.g. trays 502 and 508) may be included in the device of the present disclosure. The multiple trays 502, 508 may be stacked vertically as shown, horizontally, or in any configuration allowing for integration with a tissue dispensing apparatus.

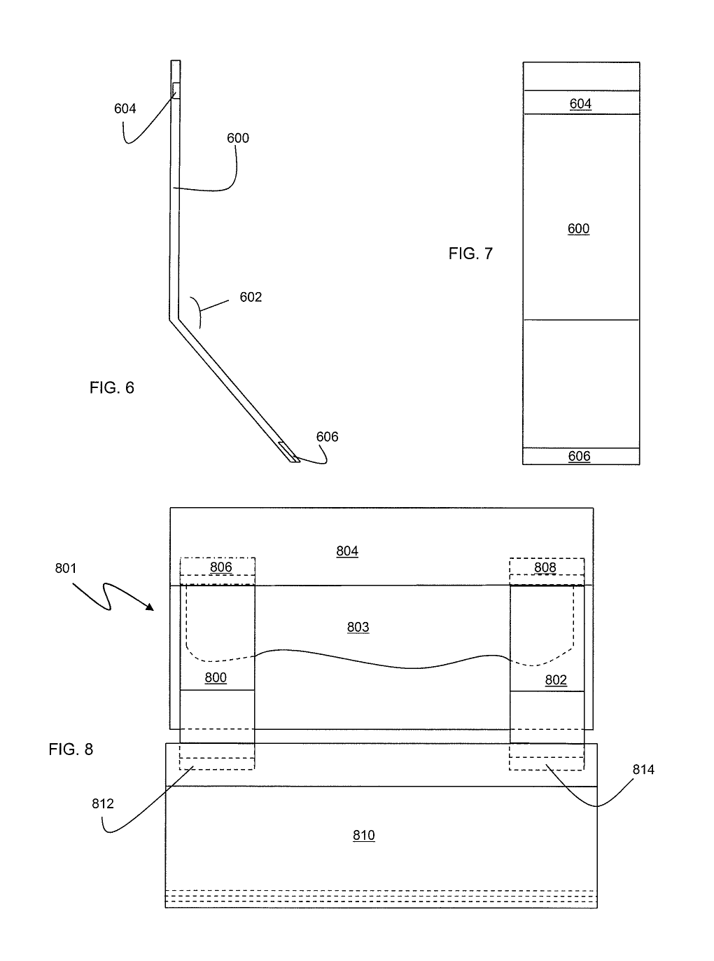

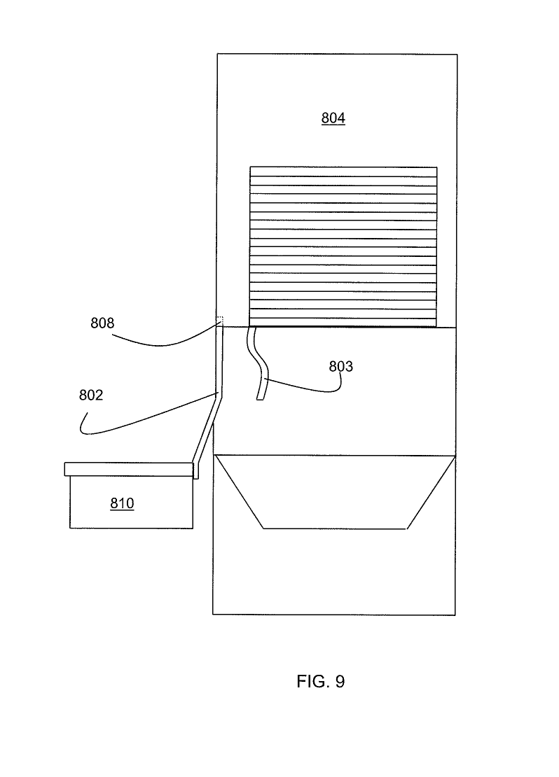

FIGS. 6 and 7 present plan and side view of a variation on the tabs disclosed above. In FIGS. 6 and 7, a tab 600 is shown having a pre-molded or formed curvature or bend 602. The tab 600 may still include an attachment component or means 604 for attaching the tab 600 (and any corresponding container) to a tissue dispensing apparatus, and an attachment component or means 606 for attaching tab 600 to a container (not shown). In this way, as further highlighted in FIGS. 8 and 9, the toiletry and sundries attachment and containment device of the present disclosure may interface and work with any type of tissue dispensing apparatus, to include those not having a roller. Further, for those dispensing apparatuses having a recessed roller or space for attaching to, the toiletry and sundries attachment and containment device can be positioned to extend beyond and out of the recessed area.

In particular, referring to the plan view of FIG. 8 and the side view of FIG. 9, one alternative embodiment of a toiletry and sundries attachment and containment device 801 of the present disclosure is presented. Tabs 800 and 802 interface with and attach to tissue (803) dispensing apparatus 804 via attachment strips or other attachment means 806 and 808 respectively. Similarly, tabs 800 and 802 interface with and attach to container or containment device 810 via attachment strips or other attachment means 812 and 814 respectively. The attachment means 806, 808 and 812, 814 may provide for either permanent, semi-permanent or removable attachment of tabs 800, 802, and hence container 810, to apparatus 804.

As best highlighted in FIG. 9, the curved nature of tabs 800 and 802 allow container 810 to extend below and beyond tissue dispensing apparatus 804, which in this case does not include a roller mechanism.

Changes may be made in the above methods, devices and structures without departing from the scope hereof. It should thus be noted that the matter contained in the above description and/or shown in the accompanying drawings should be interpreted as illustrative and not in a limiting sense. The following claims are intended to cover all generic and specific features described herein, as well as all statements of the scope of the present method, device and structure, which, as a matter of language, might be said to fall therebetween.

What is claimed is:

1. A toiletry and sundries attachment and containment device comprising:
a containing means for containing toiletry and sundries; and
an interfacing means for interfacing the containing means with a tissue dispensing apparatus;
wherein the containing means is suspended via the interfacing means in proximity to the tissue dispensing apparatus.

2. The device of claim 1, wherein the interfacing means comprises a first tab and a second tab.

3. The device of claim 2, wherein each of the first and the second tabs includes an opening for interfacing the containing means with the tissue dispensing apparatus.

4. The device of claim 1, wherein the interfacing means includes at least one attachment mechanism for attaching the interfacing means to the tissue dispensing apparatus.

5. The device of claim 4, wherein the attachment mechanism is selected from a group consisting of: a mechanical fastener, glue, Velcro®, tape, and an adhesive strip.

6. The device of claim 1, wherein the containing means is attached to the interfacing means using an attachment mechanism selected from the group consisting of: a mechanical fastener, glue, Velcro®, tape, and an adhesive strip.

7. The device of claim 1, wherein the interfacing means is integral to the containing means.

8. The device of claim 1, wherein the containing means comprises multiple trays for containing toiletries and sundries.

9. The device of claim 1, wherein the containing means is removably attached to the interfacing means.

10. A toiletry and sundries attachment device comprising:
a first tab having a first attachment mechanism for attaching to a tissue dispensing apparatus and a second attachment mechanism for attaching to a container; and
a second tab having a first attachment mechanism for attaching to the tissue dispensing apparatus and a second attachment mechanism for attaching to the container.

11. The device of claim 10, wherein the first and second attachment mechanisms of the first tab, and the first and second attachment mechanisms of the second tab, are selected from a group consisting of: a mechanical fastener, glue, Velcro®, tape, and an adhesive strip.

12. The device of claim 10, wherein the first tab and the second tab are manufactured from material selected from the group consisting of: a flexible material, semi-flexible material, semi-rigid material, and a rigid material.

13. The device of claim 10, wherein the first tab and the second tab are manufactured from a group consisting of: paper, cardboard, plastic and metal.

14. The device of claim 10, wherein at least one tab is folded during shipping and storage.

15. A toiletry and sundries attachment and containment device comprising:
a container for containing toiletries and sundries;
a first tab having a first attachment mechanism for interfacing with and attaching to a tissue dispensing apparatus, and a second attachment mechanism for interfacing with and attaching to the container; and
a second tab having a first attachment mechanism for interfacing with and attaching to the tissue dispensing apparatus, and a second attachment mechanism for interfacing with and attaching to the container.

16. The device of claim 15, wherein the container is removably attached to the first and second tabs.

17. The device of claim 15, wherein the first and the second tab are removably attached to the tissue dispensing apparatus.

18. The device of claim 15, wherein the first attachment mechanism of the first tab, and the first attachment mechanism of the second tab, are selected from a group consisting of: a mechanical fastener, glue, Velcro®, tape, and an adhesive strip.

19. The device of claim 15, wherein the second attachment mechanism of the first tab, and the second attachment mechanism of the second tab, are selected from a group consisting of: a mechanical fastener, glue, Velcro®, tape, and an adhesive strip.

20. The device of claim 15, wherein the tissue dispensing apparatus is selected from a group consisting of: a storage bin, tub, box, tray, or holder.
